(19) United States
(12) Patent Application Publication
(10) Pub. No.: US 2026/0054132 A1
(43) Pub. Date: Feb. 26, 2026

(54) APPROACHES TO PROVIDING EXERCISE THERAPY AND ADJUSTING INTENSITY IN A PERSONALIZED MANNER BASED ON SLEEP TIME, QUALITY, AND TRENDS IN THE SAME

(71) Applicant: Hinge Health, Inc., San Francisco, CA (US)

(72) Inventors: Alok Vij, Bangalore (IN); Kosanam Satish Kumar Reddy, Bangalore (IN); Sean Hellebusch, San Francisco, CA (US); Joshua Cox, San Francisco, CA (US); Hannah Skurnik, San Francisco, CA (US); Cathy Han, San Francisco, CA (US); David Cox-Espenlaub, San Francisco, CA (US); Ginson Wong, San Francisco, CA (US)

(73) Assignee: Hinge Health, Inc., San Francisco, CA (US)

(21) Appl. No.: 19/373,564

(22) Filed: Oct. 29, 2025

Related U.S. Application Data
(63) Continuation-in-part of application No. PCT/US24/43247, filed on Aug. 21, 2024.
(60) Provisional application No. 63/520,807, filed on Aug. 21, 2023.
(60) Provisional application No. 63/713,516, filed on Oct. 29, 2024.

(51) Int. Cl. A63B 24/00; A61B 5/00; A63B 71/06

(52) U.S. Cl. CPC A63B 24/0075; A61B 5/4815; A63B 71/0622; A63B 2024/0078; A63B 2214/00; A63B 2220/05; A63B 2220/806

(57) ABSTRACT

Introduced here are approaches to personalizing exercise therapy that can be implemented by a motion monitoring platform. These approaches can serve to improve personalization and variability of a physical exercise regimen, leading to improved engagement with the motion monitoring platform and improved outcomes for its users. In addition to adjusting the types of exercises to be performed by the user, the motion monitoring platform could additionally or alternatively adjust the intensity of the exercises in a personalized manner. For example, the motion monitoring platform may adjust the repetition count, hold duration, or exercise count based on an analysis of physiological data that is associated with the user, to ensure that the user does not overextend in trying to complete the series of exercises.

### CROSS-REFERENCE TO RELATED APPLICATIONS

This application claims priority to U.S. Provisional Application No. 63/713,516, titled “Exercise Therapy Personalization Based on Sleep Time, Quality, and Trends in the Same” and filed on Oct. 29, 2024, which is incorporated by reference herein in its entirety.

This application is a continuation-in-part of International Application No. PCT/US24/43247, titled “Approaches to Providing Exercise Therapy and Systems for Implementing the Same” and filed on Aug. 21, 2024, which claims priority to U.S. Provisional Application No. 63/520,807, titled “Approaches to Providing Personalized Exercise Therapy and Systems for Implementing the Same” and filed on Aug. 21, 2023, each of which is incorporated by reference herein in its entirety.

### TECHNICAL FIELD

Various embodiments concern computer programs and associated computer-implemented techniques for providing personalized physical exercises for exercise therapy.

### BACKGROUND

Exercise therapy is an intervention technique that utilizes physical exercise as the principal treatment method for addressing the symptoms of musculoskeletal (“MSK”) conditions, such as acute physical ailments and chronic physical ailments. Exercise therapy programs may involve a plan for performing physical exercises during exercise therapy sessions that occur on a periodic basis. Generally, the purpose of an exercise therapy program is to either restore normal MSK function or reduce the pain caused by an acute or chronic physical ailment, which may have been caused by injury or disease. As such, the physical exercises to be performed in each exercise therapy session may be selected in order to achieve a specific therapeutic goal. Examples of therapeutic goals include lessening pain, improving flexibility, rehabilitating injuries, managing diseases, and the like.

These exercise therapy programs normally promote engagement by starting slowly with low-intensity physical exercises and then gradually increasing the intensity over time. Such an approach helps to ensure that participants (also referred to as “patients” or “subjects”) do not “crash” by over-exercising. However, adherence to these exercise therapy programs—especially over several months or years—is difficult to maintain. For instance, patients may opt not to complete exercise therapy sessions due to boredom with the exercise therapy program, lack of noticeable improvement in acute or chronic pain, or forgetfulness. Therefore, a better approach is needed for incentivizing adherence so that patients are able to achieve lasting improvement in terms of MSK function.

### BRIEF DESCRIPTION OF THE DRAWINGS

FIG. 1 illustrates a conventional example of providing an exercise therapy program through a software-implemented motion monitoring platform.

FIG. 2 illustrates a network environment that includes a motion monitoring platform that is executed by a computing device.

FIG. 3 illustrates an example of a computing device that is able to provide a personalized exercise therapy program.

FIG. 4 includes a high-level diagrammatic illustration of a process for providing a personalized exercise therapy program, according to some embodiments.

FIG. 5 includes a diagrammatic illustration of a process for providing a personalized exercise therapy program based on anatomical region of pain, functional task, and ability level, according to some embodiments.

FIG. 6 includes a diagrammatic illustration of a process for providing a personalized exercise therapy program incorporating feedback information captured by a sensor, according to some embodiments.

FIG. 7 includes a flow diagram of a process for providing a personalized exercise therapy program, according to some embodiments.

FIG. 8 includes a flow diagram of a process for updating a series of exercises in a personalized exercise therapy program, according to some embodiments.

FIG. 9 includes a flow diagram of a process for providing a personalized exercise therapy program at a user computing device, according to some embodiments.

FIG. 10 includes a flow diagram of a process for providing a personalized exercise therapy program at a remote computing device, according to some embodiments.

FIG. 11 includes a flow diagram illustrating how different components implemented in, or accessible to, a personalization module can help in adjusting a user's exercise session based on her sleep data.

FIG. 12 includes a diagrammatic illustration showing how a scheduler can determine whether to personalize an exercise session for a user.

FIG. 13 includes a block diagram illustrating an example of a processing system in which at least some operations described herein can be implemented.

Various features of the technology described herein will become more apparent to those skilled in the art from a study of the Detailed Description in conjunction with the drawings. Various embodiments are depicted in the drawings for the purpose of illustration. However, those skilled in the art will recognize that alternative embodiments may be employed without departing from the principles of the technology. Accordingly, although specific embodiments are shown in the drawings, the technology is amenable to various modifications.

### DETAILED DESCRIPTION

Over the last several years, software-implemented platforms (or simply “platforms”) have been developed to assist in the administration of exercise therapy to patients. These platforms typically provide guidance to patients for the completion of physical exercises, as well as assisting physical therapists in education and behavioral health components. However, conventional platforms have not addressed or solved the problem of loss of patient engagement over time. Conventional platforms are generally not customizable. Instead, patients commonly utilize predetermined “playlists” of physical exercises to treat predetermined conditions, making meaningful personalization of exercise therapy difficult if not impossible. Accordingly, an individual patient may be assigned a base configuration playlist basely solely on her general condition. These base configuration playlists are rigid and inflexible, and oftentimes require manual input from physical therapist to change to reflect updated patient needs. As a result, patients on conventional platforms may feel as though an assigned physical exercise playlist is not personalized to her specific characteristics and needs. Moreover, patients may feel as through conventional platforms do not evolve with her own progress, and rather adhere to a rigid progression regardless of the performance of the individual patient in the physical exercises. Due to these sentiments, engagement oftentimes drops, resulting in less effective outcomes for patients.

Introduced here are approaches to personalizing exercise therapy that can be implemented by a motion monitoring platform. As further discussed below, these approaches can serve to improve personalization and variability of a physical exercise regimen, leading to improved engagement with the motion monitoring platform and improved outcomes for its users. Specifically, these approaches can improve user engagement over time by avoiding predetermined “playlists,” increasing variability in exercise sessions, and incorporating user feedback and objective performance information into the generated exercise program. These approaches can employ functional tasks, ability levels, and body parts to select exercises based on her characteristics. Accordingly, a series of exercises presented to each individual as a part of an exercise therapy session (or simply “session”) or an exercise therapy program (or simply “program”) that is supported, facilitated, or otherwise enabled by the motion monitoring platform will be unique based on her own functional task objectives and status, rather than a general program to treat only a specific condition.

In addition to the above, the approaches to personalizing exercise therapy provided herein allow for treatment of multiple anatomical regions of the body with a single exercise regime. In contrast to conventional approaches where each predetermined “playlist” is based solely on a condition, the approaches described herein provide for a wholistic exercise therapy program based on functional tasks and ability level. As exercises are provided to a user of the motion monitoring platform based on characteristics of the exercise and the user's desired functional task rather than provided in separate predetermined playlists for each condition, multiple body parts can be addressed with a single exercise therapy program.

Given the specific inputs of (i) anatomical region(s) of pain, (ii) functional task(s), and/or (iii) ability level, a computing device can:
Determine appropriate physical exercises for a user;
Determine an appropriate order of the physical exercises of the user;
Present a series of physical exercises to a user as a series of visualizations to guide a user through the series of physical exercises;
Provide an exercise therapy program to address multiple anatomical regions, injuries, or illnesses; and
Increase variety of exercises between exercise sessions to improve user engagement in an exercise therapy program.

As part of a program described herein, a patient may be requested to engage with the motion monitoring platform that is accessible via a computer program executing on a computing device (e.g., a mobile phone). Over time, the patient may be instructed to perform physical exercises during sessions as part of the program. For example, the patient may be instructed by an interface to perform a series of exercises over the course of a session, and the patient may be prompted to complete a series of sessions over the course of several days, weeks, or months. As discussed further below, the series of physical exercises provided may be based on input—either provided by the user or inferred, derived, or determined by the motion monitoring platform—in three categories (e.g., functional task, body part, and ability level), as well as historical exercise information and patient feedback information. The motion monitoring platform may not only assist the patient by actively guiding her through each session, but also help her adhere to the program by completing sessions in a consistent and timely manner.

As a part of some programs described herein, feedback information obtained by the motion monitoring platform may be employed to regularly update a series of exercises presented to a user of the platform, such that the series of exercises is tailored individually and provides variability in an exercise regime. This feedback information can be input directly be the user (e.g., a subjective rating of an exercise difficulty) or can be obtained from one or more sensors of the motion monitoring platform. The one or more sensors may obtain objective information regarding the performance of the patient over the series of exercises. The feedback information, whether subjective or objective, may be employed in combination with the original inputs to revise a series of physical exercises presented to the user for each session. For example, an exercise that was performed poorly may be removed from a series of exercises presented to a user. As another example, an exercise that was performed perfectly and with little to no difficult may be removed from a series of exercises, so that the exercise therapy regimen remains productive. Additional examples are discussed in detail further below. In this manner, each user of motion monitoring platform may receive a unique personalized series of physical exercises to perform for each session with the platform.

Given the feedback information of (i) sensor-obtained performance information and/or (ii) user feedback, a computing device can:
Monitor the user's progress with a sensor in real time as a series of physical exercises are completed;
Prompt the user feedback at the conclusion of a session and/or at regular intervals;
Update a series of exercises presented to a user during a session based on the feedback information;
Ensure an exercise program is tailored to a user's individual status; and
Modify an exercise program to address changes in user objectives (e.g., functional tasks) or symptoms (e.g., anatomical regions of pain).

In addition to adjusting the types of exercises to be performed by the user, the intensity of the exercises could additionally or alternatively be dynamically adjusted in a personalized manner. For example, the motion monitoring platform may adjust the repetition count, hold duration, or exercise count based on an analysis of physiological data that is associated with the user, in an effort to ensure that the user does not overextend in trying to complete the series of exercises. This is not only important in ensuring that the user does not harm herself, but also in increasing engagement as the user will feel that the series of exercises better reflects her current physical state.

Embodiments may be described in the context of computer-executable instructions for the purpose of illustration. However, aspects of the approach could be implemented via hardware or firmware instead of, or in addition to, software. As an example, the motion monitoring platform may be embodied as a computer program that offers support for completing exercises during sessions as part of a program, determines which physical exercises are appropriate for a user given performance during past sessions, and enables communication between the user and one or more coaches. The term “coach” may be used to generally refer to individuals who prompt, encourage, or otherwise facilitate engagement by users with the motion monitoring platform. Coaches are generally not healthcare professionals but could be in some embodiments.

### Terminology

References in the present disclosure to “an embodiment” or “some embodiments” mean that the feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense. That is, in the sense of “including but not limited to.” The term “based on” is also to be construed in an inclusive sense. Thus, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively connected to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing all tasks.

When used in reference to a list of multiple items, the word “or” is intended to cover all of the following interpretations: any of the items in the list, all of the items in the list, and any combination of items in the list.

### Overview

As noted above, conventional exercise therapy programs are typically rigid and based on predetermined series of exercises given to a patient over a predetermined time frame in a predetermined sequence. These programs typically include playlists of exercises for each session that are based solely on the type of injury or illness necessitating the exercise therapy. The sessions are not personalized for the individual, but rather are designed broadly for a type of injury or illness. Any adjustments to the exercise therapy program are typically a manual operation. In a software-implemented platform, such modifications must be entered manually by the patient or a physical therapist or other exercise therapy provider, which may be time consuming and difficult to manage for multiple patients.

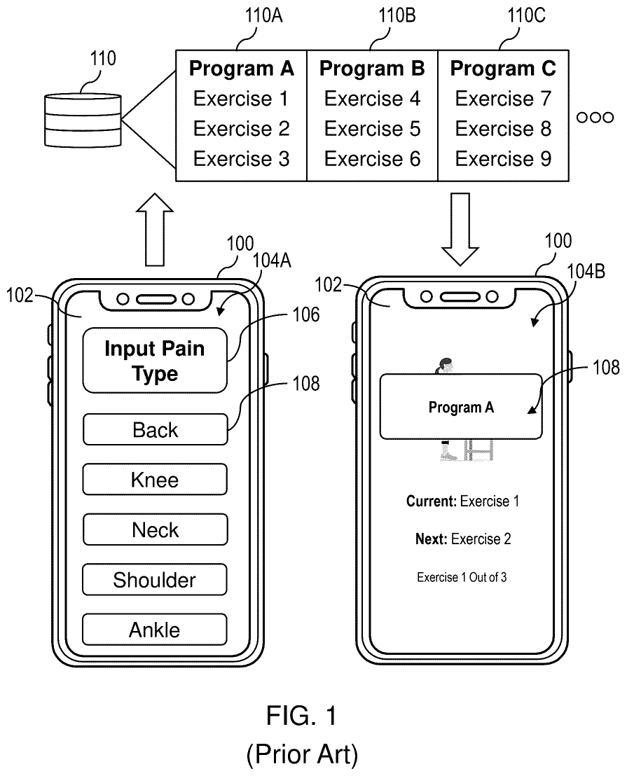

FIG. 1 illustrates a conventional example of providing an exercise therapy program through a software-implemented exercise therapy platform. Specifically, the software-implemented program of FIG. 1 is implemented on a mobile computing device 100 that includes a display 102 that is able to display interfaces and receive user input. As shown in the first interface 104A on the lower left of the figure, an input is prompted 106 from a user using the mobile computing device 100. The user is asked to specific an anatomical region of the body 108 where the pain is located. In the depicted example, the user may select from back, knee, neck, shoulder, or ankle. In other conventional examples, a user may select from a list of injuries or illnesses. For example, a user may select between options such as “chronic back pain,” “sprained ankle,” “whiplash,” and the like.

The input received from the user at the first interface 104A may, in some cases, be sent to a remote computing device that includes a database 110. In some instances, the database may be local on the computing device 100. The database may include a plurality of predetermined programs 110A-110C. Each program includes a plurality of physical exercises associated with that program. For example, a first program 110A includes three exercises: exercise 1, exercise 2, and exercise 3; a second program 110B includes three exercises: exercise 4, exercise 5, and exercise 6; and a third program 110C includes three exercises: exercise 7, exercise 8, and exercise 9. These programs may be predefined and associated with a single input from the first interface 104A. For example, back pain may be associated with the first program 110A, knee pain may be associated with the second program 110B, and neck pain may be associated with the third program 110C. The programs may be predetermined and inflexible and corresponds only to the single input at the first interface 104A.

Based on the selection at the first interface 104A, the corresponding program is retrieved from the database 110. Accordingly, multiple users of the software-implemented platform will be assigned to the same program with no variation in the exercises associated with that program. For example, as shown in a second interface 104B on the lower right, the first program 110A may be displayed on the second interface. The user may be guided through the predetermined series of exercises—in this example exercises 1-3. The series of exercises presented to a user at each subsequent session may be based on the original input at the first interface 104A. Accordingly, as the program is predetermined, the series of exercises presented at each session may be the same for all users who selected the same initial input.

### Introduction of Motion Monitoring Platform

A motion monitoring platform may be responsible for monitoring the motion of an individual (also called a “user,” “patient,” or “participant”) through analysis of digital images that contain her and are captured as she completes a physical activity such as a physical exercise. As an example, the motion monitoring platform may guide the user through exercise therapy sessions (or simply “sessions”) that are performed as part of an exercise therapy program (or simply “program”). As part of the program, the user may be requested to engage with the motion monitoring platform on a periodic basis. The frequency with which the user is requested to engage with the motion monitoring platform may be based on factors such as the anatomical region for which therapy is needed, the MSK condition for which therapy is needed, the functional task objective of the user, the difficulty of the exercise, the age of the user, the amount of progress that has been achieved, and the like.

As the user performs exercises, she may be recorded by a camera of a computing device. Normally, the camera is part of the computing device on which the motion monitoring is executed or accessed. For example, in order to initiate a session, the user may initiate a mobile application that is stored on, and executable by, her mobile phone or tablet computer, and the mobile application may instruct the user to position her mobile phone or tablet computer in such a manner that one of its cameras can record her as exercises are performed. Note that, in some embodiments, the camera is part of another computing device. For example, the camera may be included in a peripheral computing device, such as a web camera (also called a “webcam”), that is connected to the computing device. By examining the digital images that are output by the camera, the motion monitoring platform can monitor performance the exercises by estimating the pose of the user over time.

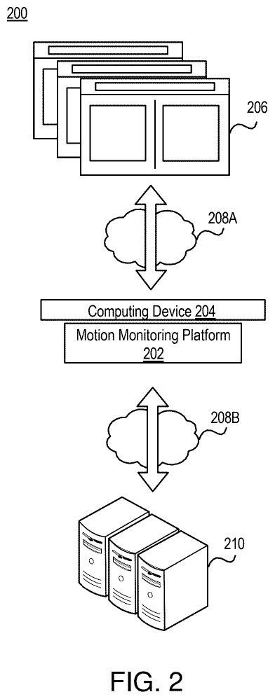

FIG. 2 illustrates a network environment 200 that includes a motion monitoring platform 202 that is executed by a computing device 204. Users can interact with the motion monitoring platform 202 via interfaces 206. For example, users may be able to access interfaces that are designed to guide them through physical exercises, indicate progress, present feedback, etc. As another example, users may be able to access interfaces through which information regarding completed physical exercises can be reviewed, feedback can be provided, etc. Thus, interfaces 206 may serve as informative spaces, or the interfaces 206 may serve as collaborative spaces through which users and coaches can communicate with one another.

As shown in FIG. 2, the motion monitoring platform 202 may reside in a network environment 200. Thus, the computing device on which the motion monitoring platform 202 is executing may be connected to one or more networks 208A, 208B. Depending on its nature, the computing device 204 could be connected to a personal area network (“PAN”), local area network (“LAN”), wide area network (“WAN”), metropolitan area network (“MAN”), or cellular network. For example, if the computing device 204 is a mobile phone, then the computing device 204 may be connected to a computer server of a server system 210 via the Internet. As another example, if the computing device 204 is a computer server, then the computing device 204 may be accessible to users via respective computing devices that are connected to the Internet via LANs.

The interfaces 206 may be accessible via a web browser, desktop application, mobile application, or another form of computer program. For example, to interact with the motion monitoring platform 202, a user may initiate a web browser on the computing device 204 and then navigate to a web address associated with the motion monitoring platform 202. As another example, a user may access, via a desktop application or mobile application, interfaces that are generated by the motion monitoring platform 202 through which she can select physical exercises to complete, review analyses of her performance of the physical exercises, and the like. Accordingly, interfaces generated by the motion monitoring platform 202 may be accessible via various computing devices, including mobile phones, tablet computers, desktop computers, wearable electronic devices (e.g., watches or fitness accessories), virtual reality systems, augmented reality systems, and the like.

Generally, the motion monitoring platform 202 is hosted, at least partially, on the computing device 204 that is responsible for generating the digital images to be analyzed, as further discussed below. For example, the motion monitoring platform 202 may be embodied as a mobile application executing on a mobile phone or tablet computer. In such embodiments, the instructions that, when executed, implement the motion monitoring platform 202 may reside largely or entirely on the mobile phone or tablet computer. Note, however, that the mobile application may be able to access a server system 210 on which other aspects of the motion monitoring platform 202 are hosted.

In some embodiments, aspects of the motion monitoring platform 202 are executed by a cloud computing service operated by, for example, Amazon Web Services®, Google Cloud Platform™, or Microsoft Azure®. Accordingly, the computing device 204 may be representative of a computer server that is part of a server system 210. Often, the server system 210 is comprised of multiple computer servers. These computer servers can include information regarding different physical exercises, including characteristics of those exercises; algorithms for processing input from a user to provide appropriate exercises to the user as well as an appropriate order of the exercises; computer-implemented models (or simply “models”) that indicate how anatomical regions should move when a given physical exercise is performed; computer-implemented templates (or simply “templates”) that indicate how anatomical regions should be positioned when partially or fully engaged in a given physical exercise; algorithms for processing image data from which spatial position of anatomical regions can be computed, inferred, or otherwise determined; user data such as name, age, weight, ailment, enrolled program, duration of enrollment, and number of physical exercises completed; and other assets.

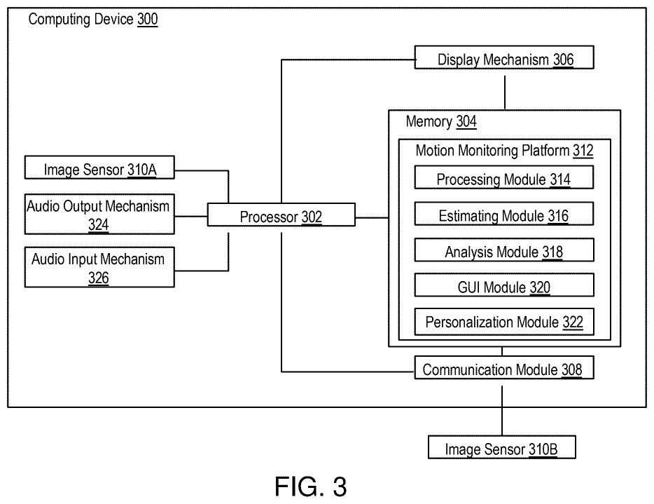

FIG. 3 illustrates an example of a computing device 300 that is able to execute a motion monitoring platform 312. As mentioned above, the motion monitoring platform 312 can facilitate the performance of physical exercises by a user, for example, by providing instruction or encouragement. Additionally, the motion monitoring platform 312 can provide a personalized series of exercises to a user to increase engagement with an exercise therapy program. As shown in FIG. 3, the computing device 300 can include a processor 302, memory 304, display mechanism 306, communication module 308, image sensor 310A, audio output mechanism 324, and audio input mechanism 326. Each of these components is discussed in greater detail below.

Those skilled in the art will recognize that different combinations of these components may be present depending on the nature of the computing device 300. For example, if the computing device 300 is a computer server that is part of a server system (e.g., server system 210 of FIG. 2), then the computing device 300 may not include the display mechanism 306, image sensor 310A, audio output mechanism 324, or audio input mechanism 326, though the computing device 204 may be communicatively connectable to another computing device that does include a display mechanism, an image sensor, an audio output mechanism, or an audio input mechanism.

The processor 302 can have generic characteristics similar to general-purpose processors, or the processor 302 may be an application-specific integrated circuit (“ASIC”) that provides control functions to the computing device 300. As shown in FIG. 3, the processor 302 can be coupled to all components of the computing device 300, either directly or indirectly, for communication purposes.

The memory 304 may be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 302, the memory 304 can also store data generated by the processor 302 (e.g., when executing the modules of the motion monitoring platform 312) and produced, retrieved, or obtained by the other components of the computing device 300. For example, data received by the communication module 308 from a source external to the computing device 300 (e.g., image sensor 310B) may be stored in the memory 304, or data produced by the image sensor 310A may be stored in the memory 304. Note that the memory 304 is merely an abstract representation of a storage environment. The memory 304 could be comprised of actual integrated circuits (also referred to as “chips”).

The display mechanism 306 can be any mechanism that is operable to visually convey information to a user. For example, the display mechanism 306 may be a panel that includes light-emitting diodes (“LEDs”), organic LEDs, liquid crystal elements, or electrophoretic elements. In some embodiments, the display mechanism 306 is touch sensitive. Thus, a user may be able to provide input to the motion monitoring platform 312 by interacting with the display mechanism 306. Alternatively, the user may be able to provide input to the motion monitoring platform 312 through some other control mechanism.

The communication module 308 may be responsible for managing communications external to the computing device 300. For example, the communication module 308 may be responsible for managing communications with other computing devices (e.g., server system 210 of FIG. 2, or a camera peripheral such as video camera or webcam). The communication module 308 may be wireless communication circuitry that is designed to establish communication channels with other computing devices. Examples of wireless communication circuitry include 2.4 gigahertz (“GHz”) and 5 GHz chipsets compatible with Institute of Electrical and Electronics Engineers (“IEEE”) 802.11—also referred to as “Wi-Fi chipsets.” Alternatively, the communication module 308 may be representative of a chipset configured for Bluetooth®, Near Field Communication (“NFC”), and the like. Some computing devices-like mobile phones and tablet computers—are able to wirelessly communicate via separate channels. Accordingly, the communication module 308 may be one of multiple communication modules implemented in the computing device 300. As an example, the communication module 308 may initiate and then maintain one communication channel with a camera peripheral (e.g., via Bluetooth), and the communication module 308 may initiate and then maintain another communication channel with a server system (e.g., via the Internet).

The nature, number, and type of communication channels established by the computing device 300—and more specifically, the communication module 308—may depend on the sources from which data is received by the motion monitoring platform 312 and the destinations to which data is transmitted by the motion monitoring platform 312. Assume, for example, that the computing device 300 is representative of a mobile phone or tablet computer that is associated with (e.g., owned by) a user. In some embodiments the communication module 308 may only externally communicate with a computer server, while in other embodiments the communication module 308 may also externally communicate with a source from which to receive image or other data (e.g., user input, sensor information, or exercise information). The source could be another computing device (e.g., a mobile phone or camera peripheral that includes an image sensor 310B) to which the mobile device is communicatively connected. Image or other data could be received from the source even if the mobile phone generates its own image or other data. Thus, image or other data could be acquired from multiple sources. In the case of image data, the data may correspond to different perspectives of the user performing a physical exercise. Regardless of the number of sources, image data, analyses of the image data, or other data may be transmitted to the computer server for storage in a digital profile that is associated with the user. The same may be true if the motion monitoring platform 312 only acquires data generated by the computing device 300 itself (e.g., image data generated by the image sensor 310A). The data may initially be analyzed by the motion monitoring platform 312, and then the data—or analyses of the data—may be transmitted to the computer server for storage in the digital profile.

The image sensor 310A may be any electronic sensor that is able to detect and convey information in order to generate images, generally in the form of image data (also called “pixel data”). Examples of image sensors include charge-coupled device (“CCD”) sensors and complementary metal-oxide semiconductor (“CMOS”) sensors. The image sensor 310A may be part of a camera module (or simply “camera”) that is implemented in the computing device 300. In some embodiments, the image sensor 310A is one of multiple image sensors implemented in the computing device 300. For example, the image sensor 310A could be included in a front- or rear-facing camera on a mobile phone. Alternatively, the image sensor 310A may be externally connected to the computing device 300 such that the image sensor 310A captures image data of an environment and sends the image data to the to the motion monitoring platform 312.

For convenience, the motion monitoring platform 312 may be referred to as a computer program that resides in the memory 304. However, the motion monitoring platform 312 could be comprised of hardware or firmware in addition to, or instead of, software. In accordance with embodiments described herein, the motion monitoring platform 312 may include a processing module 314, pose estimating module 316, analysis module 318, graphical user interface (“GUI”) module 320, and personalization module 322. These modules can be an integral part of the motion monitoring platform 312. Alternatively, these modules can be logically separate from the motion monitoring platform 312 but operate “alongside” it. Together, these modules may enable the motion monitoring platform 312 to programmatically monitor motion of users during the performance of physical exercises through analysis of digital images generated by the image sensor 310. Additionally, these modules may enable the motion monitoring platform 312 to provide to a user a personalized series of physically exercise during a session, as discussed further below.

The processing module 314 can process image data obtained from the image sensor 310A over the course of a session. The image data may be used to infer a spatial position or orientation of one or more anatomical regions as further discussed below. The image data may be representation of a series of digital images. These digital images may be discretely captured by the image sensor 310A over time, such that each digital image captured the user at different stages of performing a physical exercise. In some embodiments, these digital images may be representative of frames of a video that is captured by the image sensor 310. In such embodiments, the image data could also be called “video data.”

The image data may be used to infer a spatial position of one or more anatomical regions as further discussed below. For example, the processing module 314 may perform operations (e.g., filtering noise, changing contrast, reducing size) to ensure that the data can be handled by the other modules of the motion monitoring platform 312. As another example, the processing module 314 may temporally align the data with data obtained from another source (e.g., another image sensor) if multiple data are to be used to establish the spatial position of the anatomical regions of interest.

Moreover, the processing module 314 may be responsible for processing information input by users through interfaces generated by the GUI module 320. For example, the GUI module 320 may be configured to generate a series of interfaces that are presented in succession to a user as she completes physical exercises as part of a session. The series of interfaces may include visualizations of the exercise to be performed to guide performance of the user. On some or all of these interfaces, the user may be prompted to provide input. For example, the user may be prompted to indicate (e.g., via a verbal command or tactile command provided via, for example, the display mechanism 306) her functional task objective, subjective ability level, and anatomical region of pain. As another example, the user may be prompted to indicate that she is ready to proceed with the next physical exercise, that she completed the last physical exercise, that she would like to temporarily pause the session, etc. These inputs can be examined by the processing module 314 before information indicative of these inputs is forwarded to another module.

The pose estimating module 316 (or simply “estimating module”) may be responsible for estimating the pose of the user through analysis of image data, in accordance with the approach further discussed below. Specifically, the estimating module 316 can create, based on a digital image (e.g., generated by the image sensor 310A or image sensor 310B), a skeletal frame that specifies a spatial position of each of multiple anatomical regions. For example, the estimating module 316 can apply a computer-implemented model (or simply “model”) referred to as a pose estimator to the digital image, so as to produce the skeletal frame. In some embodiments the pose estimator is designed and trained to identify a predetermined number of joints (e.g., left and right wrist, left and right elbow, left and right shoulder, left and right hip, left and right knee, left and right ankle, or any combination thereof), while in other embodiments the pose estimator is designed and trained to identify all joints that are visible in the digital image provided as input. The pose estimator could be a neural network that when applied to the digital image, analyzes the pixels to independently identify digital features that are representative of each anatomical region of interest.

The analysis module 318 may be responsible for establishing the locations of anatomical regions of interest based on the outputs produced by the estimating module 316. Referring again to the aforementioned examples, the analysis module 318 could establish the locations of joints based on an analysis of the skeletal frame. Moreover, the analysis module 318 may be responsible for determining appropriate feedback for the user based on the outputs produced by the estimating module 316, in accordance with the approach further discussed below. Specifically, the analysis module 318 may determine an appropriate personalized recommendation for the user based on her current position, and a determination as to how her current position compares to a template that is associated with the physical exercise that she has been instructed to perform. The analysis module 318 may also determine performance scores for a user for a particular exercise. Such performance scores may be employed to provide feedback to a user and/or may be employed by a personalization module 322 to determine appropriate exercises to present to a user.

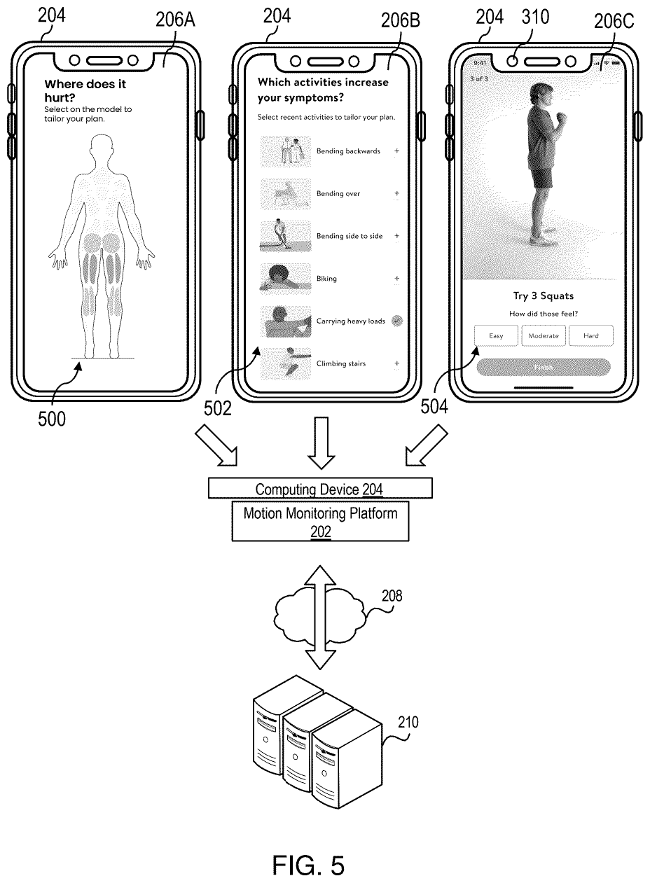

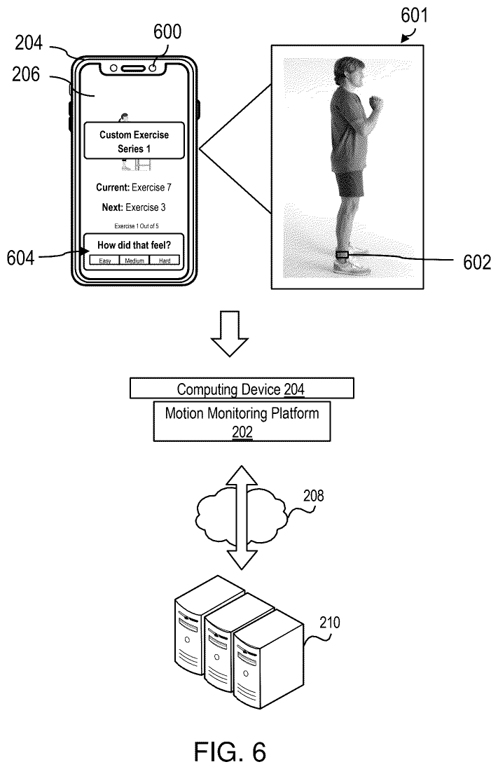

The personalization module 322 may be responsible for determining a series of exercises to be presented to the user. The personalization module 322 may be configured to receive input from a user (e.g., via a verbal command or tactile command provided via, for example, the display mechanism 306 or via the image sensor(s) 310A, 310B) that specifies (i) an anatomical region in which pain is felt, (ii) a functional task, or (iii) an ability level. Examples of these portions of the input are discussed further below with reference to FIG. 5. Based on the input, the personalization module 322 may determine appropriate physical exercises for the user to perform as a part of an exercise therapy program, as well as an appropriate order in which to perform the appropriate physical exercises. Such a determination may be based on characteristics of the exercises, such that the determined exercises and order of exercises is a part of an effective exercise therapy session. The determined exercises and order of exercises may be unique to each user and her specific input. The exercises determined by the personalization module 322 may be presented to the user (e.g., by the display mechanism 306) so that the user may be guided through a series of exercises. Feedback information may be provided to the personalization module and contribute to the determination of the appropriate exercises to present to a user as a part of an exercise therapy session. For example, image information may be employed by the analysis module 318 to determine the objective performance of the user while performing the exercises, and data representative of the performance of the user may be employed as feedback information to determine the appropriate exercises for the user. Examples of use of feedback information are discussed further below with reference to FIG. 6.

As further discussed below, the personalization module 322 may also be responsible for adjusting the intensity—for example, as measured in repetition count, hold duration, or exercise count—for the user in a personalized manner. For example, the personalization module 322 may dynamically adjust the series of exercises, or characteristics of the series of exercises, based on an analysis of physiological data that is associated with the user.

One example of physiological data is sleep data. The term “sleep data” is generally used to refer to sleep tracking data that is generated for an individual by a computing device that is worn by the individual. Sleep is most commonly monitored by measuring inactivity and movement through an analysis of measurements output by an inertial measurement unit, such as an accelerometer or gyroscope. However, various functions could be tracked, including brain and muscle activity, eye movements, heart rate, respiratory rate and airflow, blood oxygen levels, body positioning and movements, snoring, leg movements, brain waves, and sleep architecture-which is the breakdown of sleep into cycles and stages. Sleep data could be generated by the motion monitoring platform 312, for example, through an analysis of measurements output by an inertial measurement unit included in the computing device 300 or an inertial measurement unit included in another computing device to which the computing device 300 is communicatively connected. Alternatively, the motion monitoring platform 312 may obtain the sleep data from a source external to the computing device 300 or a source internal to the computing device 300 (e.g., the operating system of the computing device 300 or another computer program executing on the computing device 300).

Other examples of physiological data include heart rate data, blood glucose data, breathing rate data, and blood pressure data. While these physiological data could be generated by the computing device 300 in some embodiments, more often these physiological data are received at the communication module 308 from a source external to the computing device. Assume, for example, that the computing device 300 is a mobile phone or tablet computer. In such a scenario, physiological data could be received from another computing device—for example, a watch, fitness tracker, or continuous glucose monitor—that is regularly worn by the user. As another example, physiological data could be received from another computing device—for example, a blood glucose meter, pulse oximeter, or blood pressure monitor—that is used to measure a physiological characteristic of the user in a more ad hoc manner. Additionally or alternatively, physiological data could be received from a network-accessible storage medium that stores the physiological data and is maintained by a provider of the motion monitoring platform 312 or a healthcare system through which the user receives care. In some embodiments, rather than connect directly to a network-accessible storage medium that is maintained by a healthcare system through which the user receives care, the motion monitoring platform 312 may instead interface with (e.g., via an application programming interface) another computer program that executes on the computing device and is associated with (e.g., offered and supported by) the healthcare system.

Other modules could also be included in some embodiments. For example, the motion monitoring platform 312 may include a training module (not shown) that is responsible for training the pose estimator that is employed by the pose estimating module 316. As another example, the motion monitoring platform 312 may include a template generating module (not shown) that is responsible for generating templates that are used by the analysis module 318 to determine which recommendations, if any, are appropriate for a user given her current position.

Similarly, other components could be implemented in, or accessible to, the computing device 300 in some embodiments. For example, some embodiments of the computing device 300 include an audio output mechanism 324 and/or an audio input mechanism 326. The audio output mechanism 324 may be any apparatus that is able to convert electrical impulses into sound. One example of an audio output mechanism is a loudspeaker (or simply “speaker”). Meanwhile, the audio input mechanism 326 may be any apparatus that is able to convert sound into electrical impulses. One example of an audio input mechanism is a microphone. Together, the audio output and input mechanisms 324, 326 may enable feedback, such as personalized recommendation as further discussed below, to be audibly provided to the user. Assume, for example, that the user has been instructed to perform a physical exercise while being recorded by the image sensor 310A. In such a scenario, the user may be audibly encouraged—in a personalized manner—via the audio output mechanism 324.

### Overview of Personalization Module

As noted above, conventional approaches to exercise therapy use rigid and inflexible “playlists” that are based solely on patient condition (e.g., injury or illness). A “playlist” may include a series of physical exercises in a predetermined order. The “playlist” approach typically lays out predetermined series of exercises in a predetermined order for multiple sessions through to the conclusion of the program. In these conventional approaches, an entire exercise therapy program is provided based on a single initial input, and individual patients with the same condition are assigned the same progression of exercises regardless of individual differences in objectives and ability level. Moreover, each “playlist” is assigned to a single condition, such that a patient with multiple conditions must participate in multiple exercise therapy programs for treatment of both conditions, regardless of how the exercise programs interact with one another or include duplicative or opposing exercises.

According to exemplary embodiments herein, a computing device providing a software-implemented exercise therapy program may be configured to dynamically determine physical exercises for a user based on multiple inputs from the user. By integrating multiple inputs other than user condition, a series of exercises presented to a user may be tailored to the specific user. The inputs implemented by computing devices and methods of some embodiments described herein include (i) an anatomical region in which pain is felt, (ii) a functional task, or (iii) an ability level. A series of exercises and an appropriate order of the exercises may be determined for each exercise session following the conclusion of a preceding exercise session, rather than being part of a predetermined program. The appropriate exercises and the order of exercises may be based on assigned characteristics of the exercises. The inputs implemented by computing devices and methods of some embodiments described herein include (i) functional focus area, (ii) family, (iii) muscle chain, and (iv) exercise difficulty. The dynamically determined physical exercises may vary between each exercise session based on feedback information received directly or indirectly from a user. This feedback information may include objective information obtained by one or more sensors.

As the series of exercises and order of exercises generated by a computing device herein may be dynamic and tailored to a user, the approaches described herein overcome several disadvantages with conventional exercise therapy programs. Namely, these approaches overcome the issue of individuals struggling to adhere to the exercise therapy program. Simply put, because the dynamically determined exercise program is fully tailored and personalized, the individual will avoid becoming “immune” to exercise therapy feedback and will remain better engaged with the exercise therapy program. Moreover, a focus of the exercise program on functional task objectives allows an individual to prioritize exercises that most impact her day-to-day life.

Approaches herein employ functional tasks as an input for determining appropriate exercises to present to a user. A “functional task” refers to a specific physical activity that a user either desires to accomplish or which causes the user pain while performing the task, or both. For example, a functional task may be sitting, running, biking, playing a specific sport, climbing stairs, lifting objects, bending over, bending backwards, bending side to side, etc. Exercises may be determined based on which exercises either help the user to better accomplish task or help to avoid pain while performing the task. Data representative of one or more functional tasks may be employed as an input to a personalization module of a motion monitoring platform.

Approaches herein employ an anatomical region of pain as an input for determining appropriate exercises to present to a user. An “anatomical region” refers to a portion of the body and may include one or more muscles or muscle groups or other bodily structures. For example, an anatomical region may be the low back, which may include muscles in the lower back and upper glutes. A user may select multiple anatomical regions where pain is present, such that multiple portions of the body may be addressed by a personalized exercise therapy program. Data representative of one or more anatomical regions may be employed as an input to a personalization module of a motion monitoring platform.

Approaches herein may employ an ability level as an input for determining appropriate exercises to present to a user. An “ability level” may refer to a performance score of a user measured on a performance scale. In some embodiments, the ability level may be a single number representative of a user's ability to perform exercises generally. In some other embodiments, an ability level may be a number representative of a user's ability to perform a single exercise. In such embodiments, a “ability level” may include multiple numerical scores for different exercises. Data representative of a user's ability level may be employed as an input to a personalization module of a motion monitoring platform.

Approaches herein may associate physical exercises with one or more characteristics. A data structure that is called a “physical exercise definition” or simply “definition” may be stored in the memory and contain information about how the physical exercise is defined. The definition may include metadata about the characteristics of the exercise, which may be employed to determine which exercises are appropriate to present to a user based on user input. The characteristics may be selected from a group of functional focus area, family, muscle chain, and exercise difficulty. “Functional focus area” may be a Boolean related to whether the exercise is associated with stability or mobility. For example, each exercise may be a stability exercise or may be a mobility exercise. “Family” may be a category associated with a muscle group or movement pattern. For example, some exercises may be characterized by the spinal mobility family, lower leg mobility family, ankle mobility family, shoulder mobility family, spinal stability family, lower leg stability family, shoulder stability family and others. “Muscle chain” may be a category associated with a particular movement within a family. The muscle chain may include extension, flexion, pelvic tile, rotation, side bend, and others. “Exercise difficulty” may be a numerical score measured on a scale (e.g., 1-10, 1-50, 1-100, etc.). In some embodiments, portions of an exercise difficulty scale may be associated with difficulty “buckets” or subsets of the overall scale. For example, exercise difficulty scores on the lower half of a scale may be in a first or “easier” bucket and exercise difficulty scores on the upper half of the scale may be in a second, or “harder” bucket. An exercises difficult scale may be broken down into two or more buckets. Each individual exercise may be associated with these or other characteristics in a data structure which may be employed by a personalization module to determine appropriate exercises based on inputs received from user. In contrast to prior approaches, each exercise is associated with individual characteristics in the data structure, rather than being simply a portion of a predetermined playlist. Accordingly, the exercises may be assembled individually into a series, increasing the variety in each series, and tailoring the series to a particular user based on her inputs or feedback information.

A physical exercise definition may also include metadata about the type of exercise, preferred user state and placement within the view of the camera, and a list of heuristic conditions that define the conditions for specific state transitions within that exercise. A heuristic condition may contain some description of one or more state features (e.g., a specific pose encoded in a template, a specific joint position or flexion angle, a current state, a previous state, a time in seconds within a given state, a number of complete repetitions of the exercise, a list of other heuristic conditions), a mathematical condition on those state features (e.g., the value of the feature or some metric determined from the feature being less than, equal to, or greater than a threshold, a comparison between the values of two state features), or a score that may be based on the degree of acceptance of the aforementioned mathematical condition and may be used to rank valid conditions.

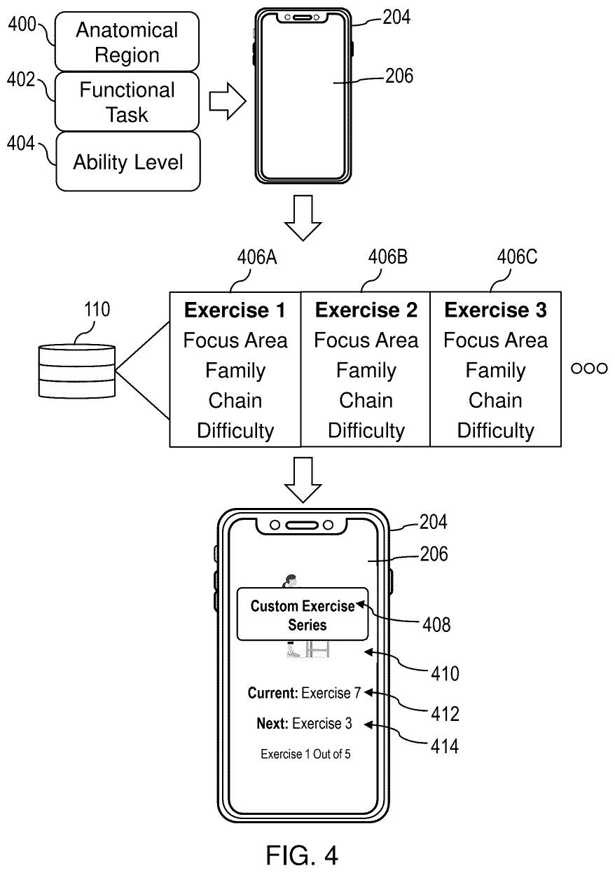

FIG. 4 includes a high-level diagrammatic illustration of a process for providing a personalized exercise therapy program, according to some embodiments. As shown in FIG. 4, a software-enabled exercise therapy program may be implemented on a computing device 204 which includes an interface 206 which is able to display information and receive user input. In the example of FIG. 4, the computing device 204 is a mobile phone. The computing device 204 may be configured to receive input from a user. Specifically, an interface on the interface 206 may prompt the user to provide input, for example by pressing a button.

In the example of FIG. 4, a user is prompted to provide three inputs. A first input 400 is an anatomical region of the body in which pain is felt. A second input 402 is a functional task which the user of the computing device 204 wishes to accomplish or a task that causes the user pain. A third input 404 is an ability level. These inputs may be received by the computing device via direct user input, in some embodiments. For example, a user may be guided through an interface where a user selects affected anatomical regions of the body as the first input, selects functional tasks, and inputs an ability level as a general score or for individual exercises. In some embodiments, the computing device may receive the user input indirectly. For example, a user may be prompted to perform test exercises and a sensor may capture information while the user completes the test exercises. This sensor information may be employed to inferred or otherwise compute an ability score based on the test exercises.

The three inputs received at the computing device 204 are employed to determine appropriate exercises and an appropriate order of those exercises. The input received from the user at the computing device 204 may, in some cases, be sent to a remote computing device which includes a database 110. In some instances, the database may be local on the computing device 204. The database may include a plurality of exercises 406A-406C. Each exercise includes a plurality of characteristics stored in a data structure. For example, a first exercise 406A has an assigned focus area, family, chain, and difficult level. Likewise, each of a second exercise 406B and a third exercise 406C has an associated focus area, family, chain, and difficulty level. While three exercises are show, this general exercise structure may extend to tens, hundreds, or thousands of exercises stored within the database 110.

The remote computing device or the computing device 204 may determine a subset of the exercises stored on the database 110 based on the input received from the user. That is, an algorithm executed by a processor f the computing device or remote computing device may determine which exercises are appropriate for the inputs received and what order of exercises is appropriate for the inputs received. Exercises may be evaluated individually based on the inputs to determine if those exercises are most appropriate to the received inputs. Exercises having a best score according to the inputs may selected as a part of a series of exercises presented to the user. Guidelines may guide the determination of the exercises based on the user input. For example, the number of exercises may be between 3 and 12 exercises, 4 and 10 exercises, or 5 and 7 exercises. In some embodiments, the exercises may be between 5 and 7 exercises, which may improve user engagement over time while retaining exercise therapy effectiveness. As another example, at least two physical exercises included in a series may be different from physical exercises previously provided to a user as part of an exercise session. As still another example, exercises may be included in a series based on individual difficulty or overall average exercise difficulty of the series. In some such examples, later series may have a different (e.g., higher, or lower) average exercise difficultly than an earlier series. As still another example, exercises may be included as a part of a series so that both stability focus area exercises and strength focus area exercises are included. In some cases, stability focus area and strength focus area exercises may be included in a ratio between 3:5 and 5:3. Simply put, a series of exercises may be determined from a plurality of exercises and the characteristics of those exercises based on input received from a user. The series of exercises may therefore be personalized to the user based on the user input. The series of exercise may then be presented to the user.

The series of exercises determined based on the first input 400, second input 402, and the third input 404 may be presented to the user on the interface 206 of the computing device 204. As shown in FIG. 4, a customer exercise series 408 may be displayed as a part of the interface 206. The interface may indicate a current exercise to perform 412 and may assist the user in performing the exercise with a diagram 410, instructions, video, or other information. The interface may also indicate the next exercise 414 on the interface, in some cases. Notably, the exercises shown in the interface of FIG. 4 are not a part of a predetermined playlist but are rather determined for inclusion in the customer exercise series 408 based on the inputs received from the user and the characteristics of the exercises, as discussed above. During the performance of exercises feedback information may be from the user, examples of which are discussed further below with reference to FIG. 6.

FIG. 5 includes a diagrammatic illustration of a process for providing a personalized exercise therapy program based on anatomical region of pain, functional task, and ability level, according to some embodiments. In the example of FIG. 5, a computing device 204 including an interface 206 is implemented to receive user input. The computing device 204 may implement a personalization module as a part of a motion monitoring platform 202. The computing device 204 may reside in a network environment 200. The computing device may be connected to a network 208. In the depicted example, the computing device 204 is connected to a remote computing device of a server system 210 via the Internet. Data may be sent to a remote computing device of the server system 210 or received from the remote computing device of the server system 210, for example, via a communications module.

The computing device 204 is configured to receive user input via interfaces 206A-206C. In a first interface 206A, the computing device receives first user input 500 regarding an anatomical region where pain is felt. In some embodiments as shown in FIG. 5, a model of the body may be depicted on the first interface, and a user may select different regions of the body where pain may be present. The first user input may include at least one anatomical region as a list. The list of anatomical regions may be sent to the server system 210. Accordingly, the server system 210 may determine exercises based on the precise anatomical regions where the user has pain, rather than a general condition. Additionally, a series of exercises may address pain in multiple anatomical regions.

In a second interface 206B, the computing device receives second user input 502 regarding functional tasks. In the example of FIG. 5, activities which increase symptoms (e.g., pain) are selected by the user. In some embodiments, the user may select functional tasks from a list. The list of functional tasks may be sent to the server system 210. Accordingly, the server system 210 may determine exercises based on which tasks increase a user's pain. A series of exercises determined by the server system 210 may address multiple functional tasks.

In a third interface 206C, the computing device receives third user input 504 regarding ability level. In the example of FIG. 5, a user is prompted to perform test exercises. In some embodiments, the user may provide a subjective rating of how the exercises felt. The subjective rating may be a numerical value measured on a scale, in some embodiments. The ability level value may be sent to the server system 210. Accordingly, the server system 210 may determine exercises based on the user's personal ability level. A series of exercises determined by the server system 210 may be suited to this ability level so that the user receives exercises that are not too difficult to too easy. In some embodiments, the ability level may be inferred or computed based on information received from an image sensor 310 of the computing device 204. For example, images obtained by the image sensor 310 while the user is performing the test exercises may be used to determine an ability level in performing those text exercises. In such examples, the ability level may be assigned to the user by the computing device. In some embodiments, a combination of subjective user input and information obtained from sensors may be employed to determine an ability level for an exercise or an overall ability level.

FIG. 6 includes a diagrammatic illustration of a process for providing a personalized exercise therapy program incorporating feedback information captured by a sensor, according to some embodiments. In the example of FIG. 6, a computing device 204 including an interface 206 is implemented to obtain feedback information. The computing device 204 may implement a personalization module as a part of a motion monitoring platform 202. The computing device 204 may reside in a network environment 200. The computing device may be connected to a network 208. In the depicted example, the computing device 204 is connected to a remote computing device of a server system 210 via the Internet. Data, including the feedback information, may be sent to a remote computing device of the server system 210 or received from the remote computing device of the server system 210, for example, via a communications module. This data may be employed to determine a series of exercises to present to a user.

The computing device 204 is configured to receive feedback information from one or more sensors. In some embodiments as shown in FIG. 6, the computing device 204 includes an image sensor 600 configured to capture images 601 of the user while the user is performing an exercise. In the depicted embodiment the image sensor 600 is a part of the computing device 204, but in other embodiments may be a part of another device in communication with the computing device 204. The images 601 captured by the image sensor 600 may be captured in real time, and in some cases may be used to determine the performance of a user in a variety of exercises. For example, a pose of the user may be determined from captured images and compared to a desired pose. Such a comparison may be used to determine a performance score for a particular exercise or an overall performance score for a series of exercises. In some cases, determination of pose from images 601 may be completed onboard the computing device 204. In other embodiments, the images may be sent to a remote computing device of the server system 210 to be processed. The performance score(s) determined based on the images 601 may be used to determine a series of exercises for the next exercise therapy session. For example, if a user has an overall performance score below a threshold, exercises with an easier difficulty may be prioritized for inclusion in the next series. As another example, if a user has an overall performance score above a threshold, exercises with a harder difficult may be prioritized for inclusion in the next series. As still another example, if a user has an individual exercise performance score below a performance threshold, that exercise may be removed from the next series to avoid improper form. The feedback information may be implemented in a variety of algorithms to ensure that a series of exercises presented to a user reflect the user's most current performance status.

In some embodiments, other optional external sensors may be implemented with a computing device 204 to obtain feedback information. For example, a wearable sensor 602 such an accelerometer may be worn on one or more body parts during the performance of a series of exercises. Data obtained from external sensors may supplement or replace image information in some cases.

The computing device 204 is configured to receive feedback information via an interface 206. In some embodiments as shown in FIG. 6, the interface 206 may be an interface guiding a user through a series of exercises. An interface 206 of the computing device 204 may also prompt input of direct user feedback 604 (e.g., with a button, slider, etc.). For example, a user may indicate how a particular exercise felt, or how a particular series of exercises felt. In some cases, feedback may be a numerical score measured on a scale. The feedback input received from a user may also be employed to determine a subsequent series of exercises, so that the exercises can be updated to reflect the user's sentiments regarding their own performance. In this manner, exercises that a user feels more positively about may be prioritized to ensure continued engagement with the exercise program.

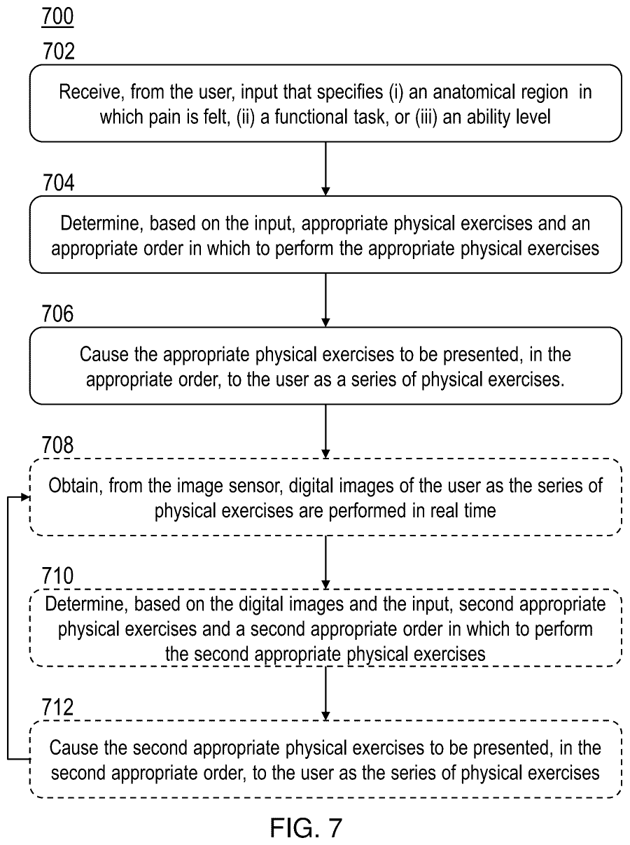

FIG. 7 includes a flow diagram of a process 700 for providing a personalized exercise therapy program, according to some embodiments. In step 702, input is received from a user that specifies (i) an anatomical region in which pain is felt, (ii) a functional task, or (iii) an ability level. In some embodiments, the input may include an anatomical region, a functional task, and an ability level. In step 704, appropriate physical exercises and an appropriate order of the physical exercises are determined based on the input. The determination may be algorithmic and based on comparison(s) of the input to one or more characteristics of the physical exercises. In some embodiments, a trained statistical model may be employed to determine an output of a series of exercises for a given input. In some embodiments, the determination may be performed on a remote computing device, where a local computing device transmits the input to the remote computing device and receives the appropriate exercises in the appropriate order from the remote computing device. In step 706, the process may include causing the appropriate physical exercises to be presented in the appropriate order to a user as a series of physical exercises. In some embodiments, the series of exercises may be presented as a part of an interface on a display of a computing device.

The process 700 includes optional steps for updating a series of exercises presented to a user to provide an exercise program. In optional step 708, the process includes obtaining digital images from an image sensor. The digital images include the user performing the series of physical exercises in real time. In some cases, the digital images may be analyzed to determine a performance score of the user. In some cases, the digital images may be processed to determine a pose of various anatomical regions of the user, and the pose may be compared to a desired pose for a particular exercise. In step 710, the process includes determining second appropriate exercises and a second appropriate order for the exercises based on the digital images and the original input. For example, exercises that are not performed well (with performance scores falling below a first threshold) may be replaced by other exercises that still accomplish improvement of the functional task in a desired anatomical region. Exercises that are performed too well (with performance scores falling above a second threshold) may be too easy and replaced by other exercises that allow a user to further progress. In step 712, the process includes causing the second appropriate physical exercises to be presented to the user in the second appropriate order. Steps 708-712 may be repeated for each presentation of a series of physical exercises until an exercise program is completed. In this manner, the series of exercises presented to a user may change and evolve over time according to the user's actual progress as well as their original objectives in entering the program.

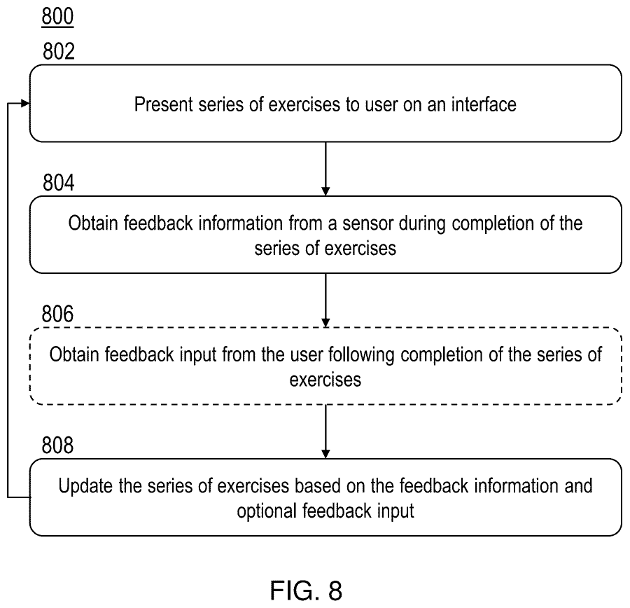

FIG. 8 includes a flow diagram of a process 800 for updating a series of exercises in a personalized exercise therapy program, according to some embodiments. In step 802, the process includes presenting a series of exercises to a user on an interface of a computing device. In step 804, the process includes obtaining feedback information from a sensor during completion of the series of exercises. The obtaining of feedback information from a sensor may be like the approach described with reference to FIG. 6. In step 806, the process includes obtaining feedback input from a user following completion of the series of exercise. The feedback input may be received at the interface in some embodiments, for example as shown in FIG. 6. Step 806 may be optional, in some embodiments. In step 808, the process includes updating the series of exercises based on the feedback information and the optional feedback input. The updating of exercises may be similar to the processes described with respect to FIGS. 6 and 7. The process of FIG. 8 may be repeated for each presentation of a series of physical exercises until an exercise program is completed. In this manner, the series of exercises presented to a user may change and evolve over time according to the user's actual progress as well as their subjective opinion on the exercises.

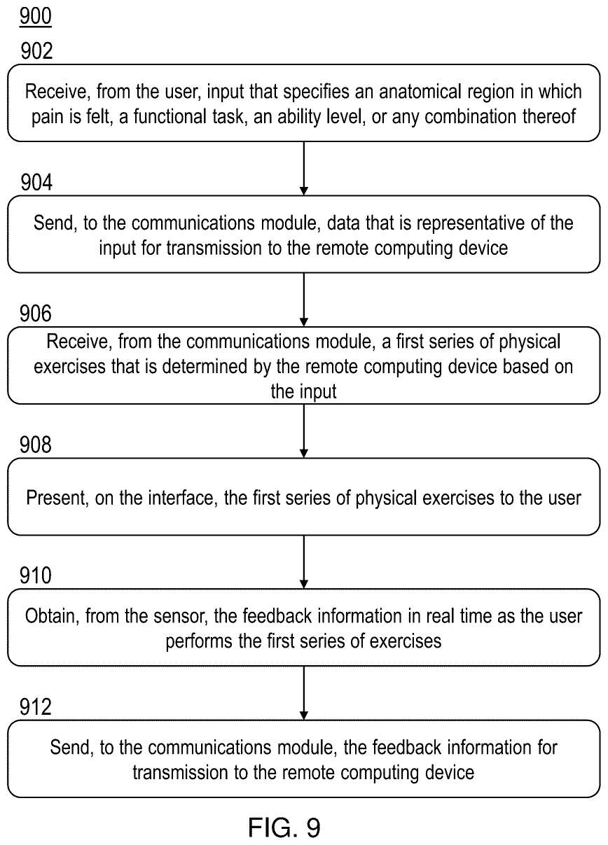

FIG. 9 includes a flow diagram of a process 900 for providing a personalized exercise therapy program at a user computing device, according to some embodiments. In step 902 the process includes, receiving, from a user at an interface on the computing device, input that specifies an anatomical region in which pain is felt, a functional task, or an ability level. The user may provide the input via a tactile selection, in some embodiments. In some embodiments, the input may include at least a functional task and an anatomical region. In step 904, the process includes sending data that is representative of the input to a communications module (e.g., see communications module 308 in FIG. 3) for transmission to the remote computing device. The remote computing device may determine a series of exercises to present to a user based on the input, in this embodiment. In step 906, the process includes receiving, from the communications module, a first series of physical exercises. The first series of physical exercises may be generated by the remote computing device dynamically based on the input. For example, the remote computing device may apply an algorithm to the input that determines exercises that correspond to the input based on the characteristics of the exercises. In step 908, the process includes presenting on an interface the first series of physical exercises to the user. The first series of exercises may be presented on the interface of the computing device.

In step 910 of the process 900, feedback information is obtained from a sensor in real time as the user performs the first series of exercises, as guided by the computing device. The sensor may include an image sensor that captures images of the user. In other embodiments, the sensor may include a wearable sensor such as an accelerometer. In step 912, the feedback information is sent to the communications module for transmission to the remote computing device. The remote computing device may integrate the feedback information into a subsequent determination of a series of exercises to present to the user.

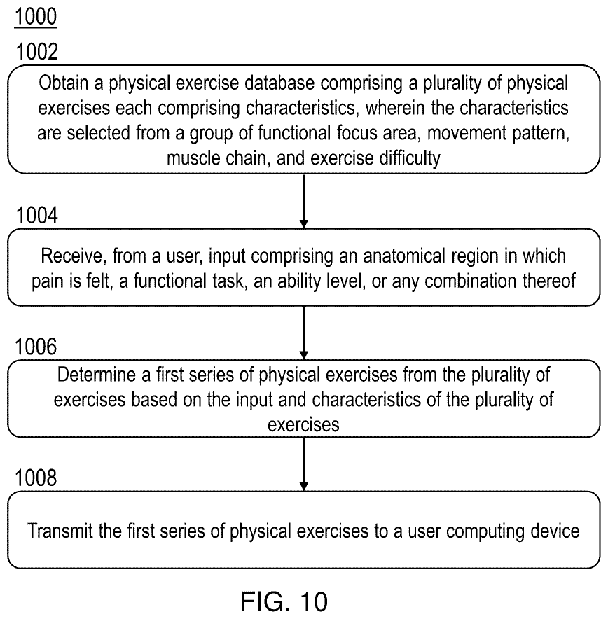

FIG. 10 includes a flow diagram of a process 1000 for providing a personalized exercise therapy program at a remote computing device, according to some embodiments. In step 1002, the process includes obtaining a physical exercise database comprising a plurality of physical exercises each comprising characteristics. The characteristics may be selected from the group of functional focus area, family, muscle chain, and exercise difficulty. In step 1004, the process includes receiving input from a user comprising an anatomical region in which pain is felt, a functional task, an ability level, or any combination thereof. The input may be input by the user on an interface of a computing device (or may be otherwise capture by a computing device) which transmits the input to the remote computing device via one or more networks. In step 1006, a first series of physical exercise is determined by the remote computing device based on the input. The first series of exercises may be a subset of the plurality of physical exercises stored in the database. In some embodiments, the first series of physical exercises includes between 3 and 12 exercises, 4 and 10 exercises, or 5 and 7 exercises. In step 1008, the process includes transmitting the first series of physical exercise to a user computing device (e.g., the computing device that originally received the input). The first series of exercises may be transmitted by one or more networks.

In some embodiments, the remote computing device may store the first series of exercises and associate the series with a user profile. In such embodiments, feedback information and further input may be received from a computing device and stored in the user profile. A user profile may also include an overall performance score and/or performance scores associated with each exercise. As subsequent series of exercises are generated, the remote computing device may incorporate information stored in the user profile. For example, while generating a second series of exercises, at least two physical exercises in the second series of exercises may be different from the first series of physical exercises. A user profile may ensure a user's progress can be monitored and the determined exercises are most likely to retain user engagement with an exercise therapy program.

### Approaches to Personalized Adjustment Based on Physiological Data

In addition to adjusting the types of exercises to be performed by the user, the personalization module 322 may also be able to dynamically adjust the intensity of the exercises in a personalized manner. For example, the personalization module 322 may adjust the repetition count, hold duration, or exercise count based on an analysis of physiological data that is associated with the user, in an effort to ensure that the user does not overextend in trying to complete the series of exercises. This is not only important in ensuring that the user does not harm herself, but also in increasing engagement as the user will feel that the series of exercises better reflects her current physical state. The following embodiments are described in the context of sleep data for the purpose of illustration. Those skilled in the art will recognize that the features of these embodiments may be similarly applicable to other forms of physiological data, such as heart rate data, blood glucose data, breathing rate data, and blood pressure data.

Numerous studies have shown that inadequate sleep or acute sleep lose will affect physical performance. This effect is observed across all exercise categories, such as anaerobic power, speed endurance, power endurance, high-intensity interval exercise (also called “HIIE”), strength, endurance, and strength-endurance. Lack of sleep also tends to result in decreased motivation, and therefore leads to users skipping exercise sessions. The effect is even more pronounced with increased age, which may constitute a significant population of the users that are completing exercise sessions for physical therapy purposes.

Today, many modern computing devices and peripherals provide information related to sleep quality and/or duration (or information from which insights into sleep quality and/or duration are derivable). For example, the HKCategoryValueSleepAnalysis library available on computing devices manufactured by Apple Inc. provides comprehensive sleep data, including information on total sleep, rapid eye movement (“REM”) sleep, core sleep, and deep sleep. Introduced here is an approach in which sleep data is used to control and change the intensity of exercises to be performed during an exercise session. For example, based on sleep data, the motion monitoring platform—and more specifically, its personalization module—can adjust the difficulty of an exercise session so that the user can complete the exercise session according to her current condition. These adjustments can include removing more strenuous exercises, replacing more strenuous exercises with less strenuous exercises, decreasing the number of repetitions required for at least one of the exercises to be performed during the exercise session, decreasing a range of motion required by at least one of the exercises to be performed during the exercise session, and the like.

This will allow users to engage with the motion monitoring platform according to their physical capacity to perform exercises on that day. Moreover, it will allow users to remain consistent and motivated for exercise sessions as the users will know that the exercises can be adjusted based on current capacity for physical activity and overall wellness.

Through research, a clear pathway on how sleep data can be used to adjust exercise therapy for a given user has been established. For purposes of illustration, a framework has been developed that implements a linear relationship between sleep duration loss and exercises using difficulty, repetition count, exercise family, and exercise chain. A part of a sample output is presented below in Tables I and II. Assume, for example, that the set of exercises to be performed by a user includes those exercises listed in Table I. In response to a determination—based on an indication from the user or analysis of sleep data associated with the user—that the total sleep duration loss is 25 percent, the set of exercises can be adjusted as shown in Table II. In this example, the exercises and number of repetitions have been adjusted, though that need not be the case. Difficulty could be decreased by simply decreasing the number of repetitions, or by replacing one exercise with another less difficult exercise but requiring the same number of repetitions. The personalization module could also decrease difficulty by replacing an exercise in one exercise family with another exercise in another less difficult exercise family.

TABLE I
Set of exercises initially defined for a user
in accordance with an exercise therapy program.

| Initial | | | | |
|---|---|---|---|---|
| Exercise Name | Family | Chain | Difficulty | Reps |
| Bear Crawl Holds | Anterior Core Strength | Planks | 60 | 4 |
| Hollow Hold | Anterior Core Strength | Hollow | 75 | 4 |
| | | Hold | | |
| Alternating | Lower Body Spinal | Pelvic | 30 | 4 |
| Pelvic Tilts | Mobility | Tilt | | |
| Prone Press Up | Lower Body Spinal | Extension | 50 | 4 |
| | Mobility | | | |

TABLE II
Set of exercises as adjusted to account
for loss of total sleep duration.

| Updated | | | | |
|---|---|---|---|---|
| Exercise Name | Family | Chain | Difficulty | Reps |
| Plank on Knees | Anterior Core Strength | Planks | 50 | 3 |
| Half Bent | Anterior Core Strength | Hollow | 55 | 3 |
| Hollow Hold | | Hold | | |
| Alternating | Lower Body Spinal | Pelvic | 30 | 3 |
| Pelvic Tilts | Mobility | Tilt | | |
| Wall Backed | Lower Body Spinal | Extension | 50 | 3 |
| Extensions | Mobility | | | |

The foundational concept of leveraging sleep data to optimize users' exercise therapy can be expanded by analyzing sleep patterns and trends, enhancing overall user care. The following mechanisms could be employed to further expand the foundational concept:
By examining sleep duration and/or quality, the personalization module can identify patterns of “social jetlag” and tailor exercise therapy accordingly. The term “social jetlag” is commonly used to refer to significant contrasts in sleep patterns between weekdays and weekends (e.g., where duration changes by more than an hour). For example, if a user tends to sleep more on Fridays and Saturdays, her exercise sessions for Fridays and Saturdays could be intensified, capitalizing on the increased rest and motivation in advance. Conversely, her exercise sessions for Sundays could be made easier if the personalization module is able to establish that she routinely sleeps less on Saturdays. Thus, exercise sessions could made more difficult for days that users are expected to be generally more rested and made less difficult for days that users are expected to be generally less rested.
Through sleep pattern analysis, automated goals or action plans could be formulated for the days that users are mostly likely to achieve those goals or complete those action plans, thereby boosting exercise therapy engagement.
Displaying sleep pattern data to members of a “care team”—like physical therapists, for example—may enable those individuals to provide targeted recommendations, ensuring a more personalized and effective strategy for providing care.

Additionally, the utilization of sleep data holds promise for not only enhancing user engagement, but also for optimizing pain management strategies within the exercise therapy programs. There exists a strong association between sleep patterns and musculoskeletal pain. Based on sleep data and analyses thereof, especially when extended periods of light sleep or inconsistent sleep are observed, exercise therapy can be adjusted to focus on improving sleep, not simply improving exercise session performance. Such an approach aims to alleviate users' pain the following manner:
For users determined to be experiencing sleep difficulties based on an analysis of sleep patterns, pre-sleep routines such as light exercises, meditative sessions, and relaxing music could be incorporated as components of the exercise session to improve sleep duration and quality. These routines may also be recommended selectively on the basis of social jetlag.
Based on sleep data insights, as well as pain area indications input through interfaces generated by the GUI module 320, tailored recommendations can be made regarding optimal sleep positions, such as side sleeping with a pillow between the legs for users who are experiencing back pain.
Inconsistent sleep data could be shared with members of the care team. If the recommendations do not help in alleviating sleep problems, care team members may recommend consultations with a healthcare professional (e.g., a somnologist). This holistic approach can help users optimize sleep quality, and thereby focus on reducing pain and extracting maximum benefits through the motion monitoring platform.

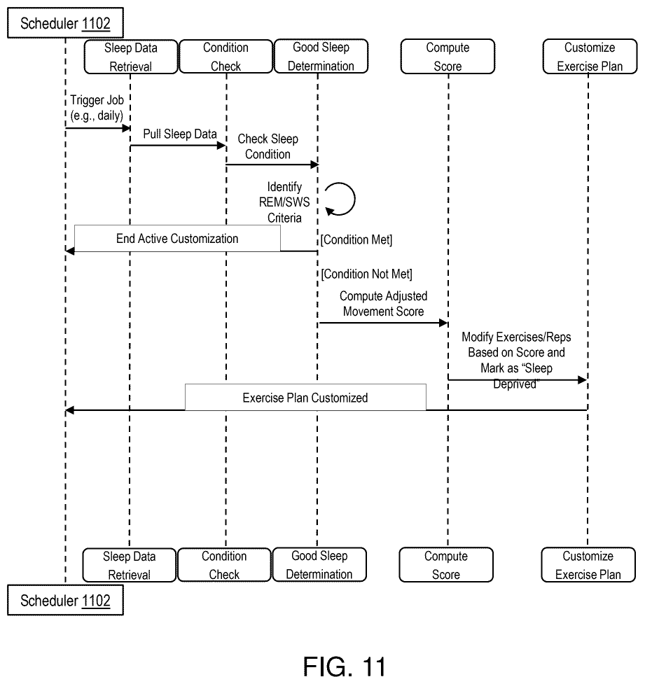

FIG. 11 includes a flow diagram illustrating how different components implemented in, or accessible to, the personalization module 322 can help in adjusting a user's exercise session based on her sleep data.

At a high level, this process begins with a scheduling component (also called a “scheduling module” or the “scheduler”) 1102 triggering the retrieval of sleep data from the memory 304 or from another computing device (e.g., a mobile phone, watch, or fitness tracker). In some embodiments, the scheduler 1102 does this periodically, for example, on a daily basis. In other embodiments, the scheduler 1102 does this in a more ad hoc manner. For example, the scheduler 1102 may trigger retrieval of sleep data in response to a determination that the user has initiated the motion monitoring platform 312 or otherwise indicated an interest in completing an exercise session. Referring to FIG. 3, the scheduler 1102 may be part of the motion monitoring platform 312 but distinct from the processing module 314, estimating module 316, analysis module 318, GUI module 320, and personalization module 322. Alternatively, the scheduler 1102 may be part of the personalization module 322.

Depending on whether the member achieves a predetermined sleep condition, the personalization module 322 can either maintain or modify the set of exercises to be completed as part of an exercise session. The sleep condition can be defined by thresholds (e.g., for total sleep duration, REM sleep, core sleep, deep sleep, or slow wave sleep). These thresholds may vary based on the user's age and may be guided by an age-related trends graph. Alternatively, these thresholds could be defined for each user, for example, based on an analysis of sleep data corresponding to a preceding interval of time (e.g., one month, three months, six months).

If the sleep condition is not met, the personalization module 322 can compute an adjusted movement score and revise the exercise session accordingly, for example, by excluding exercises with difficulty levels exceeding the adjusted movement score or decreasing repetition count until the difficulty level falls below the adjusted movement score. Moreover, the personalization module 322 can label the user as “sleep deprived” by populating an indication of that status in a data structure maintained for the user. The data structure could be stored in the memory 304, or the data structure could be stored in a memory external to the computing device 300—in which case the indication may be forwarded to the communication module 308 for transmittal to the destination.

If the sleep condition is met, then the personalization module 322 may take no action—allowing the exercise session to proceed without changes. Further, the personalization module 322 may remove any customizations made for a prior “sleep deprived” status, so as to revert to the standard exercise therapy for future exercise sessions.

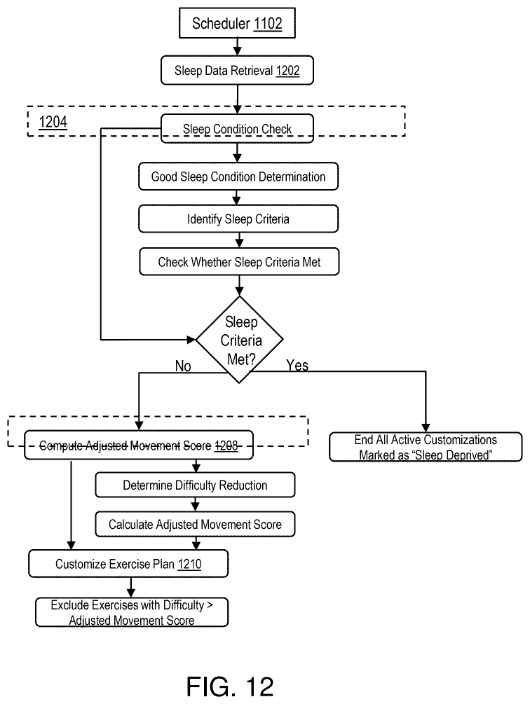

FIG. 12 includes a diagrammatic illustration showing how the scheduler 1102 can determine whether to personalize an exercise session for a user. As mentioned above, the scheduler 1102 may be responsible for periodically triggering a job (e.g., that runs on a daily basis at a predetermined time) to retrieve sleep data for the user. Preferably, the scheduler 1102 may trigger the job after the user is most likely to have finished sleeping, such that the latest sleep data is retrieved. In some embodiments, the job is triggered by the scheduler 1102 in response to a determination that a predetermined temporal criterion has been met. For example, the scheduler 1102 may trigger the job at a given time (e.g., 7 AM, 8 AM, 9 AM) every morning through a comparison of a clock signal generated by the computing device 300 to a predetermined time that is representative of a temporal criterion. In other embodiments, the job is triggered by the scheduler 1102 in response to an indication that the user has woken up. For example, if another computer program executing on the computing device 300 or the operating system of the computing device 300 indicates that the user has performed an access event (e.g., entered a password, scanned a fingerprint, scanned a face), then the scheduler 1102 may infer that the user has awoken and then trigger the job.

In step 1202, the motion monitoring platform 312 automatically pulls sleep data that is associated with the user. For example, the motion monitoring platform 312 may retrieve the sleep data from another computing device (e.g., a watch or fitness tracker) to which the computing device 300 is communicatively connected. The sleep data can include various information, such as total sleep duration, sleep stages, interruptions, and possibly other metrics like heart rate variability. This information can be used to evaluate sleep quality and then determine whether adjustments to the exercise session are necessary.

In step 1204, the personalization module 322 can perform a sleep condition check. If the personalization module 322 determines, based on an analysis of the sleep data, that the user does not satisfy a sleep condition, the personalization module 322 can (i) compute an adjusted movement score and (ii) customize the exercise session to account for the poor sleep. As mentioned above, the exercise session could be customized be excluding exercises with a difficulty level greater than the adjusted movement score or adjusting the exercises (e.g., by decreasing repetition count or lessening range of movement) until the difficulty level falls below the adjusted movement score. Moreover, the personalization module 322 can mark the user as “sleep deprived,” for example, by indicating that status in a data structure that is maintained for the user. The data structure may be representative of a user profile. If the personalization module 322 determines, based on an analysis of the sleep data, that the user does satisfy the sleep condition, the personalization module 322 can clear any active customizations if the user is marked as having a “sleep deprived” status and refrain from customizing the exercise session any further.

In step 1206, the personalization module 322 can determine whether the user satisfies a good sleep condition check. More specifically, the personalization module 322 can compare one or more sleep characteristics—which may be specified in the sleep data or calculated by the personalization module 322—satisfy corresponding criteria. In FIG. 12, for example, the personalization module 322 determines whether REM sleep satisfies a corresponding criterion and whether slow wave sleep (“SWS”) satisfies a corresponding criterion. Other sleep characteristics—such as total sleep duration, eye movement, heart rate, respiratory rate and airflow, blood oxygen levels, body positioning and movements, snoring presence, onset variance, wake variance, and the like—could be used instead of, or in addition to, REM sleet and SWS. The criteria for these sleep characteristics can be identified based on the user's age from age-related trends graphs or determined in a more personalized manner, as discussed above.

In step 1208, the personalization module 322 can compute the adjusted movement score. At a high level, the purpose of computing the adjusted movement score is to quantify the degree to which the exercise session—or individual exercises—should be modified to account for the current physical state of the user. As an example, the personalization module 322 may use a difficulty reduction table that indicates, based on scores for one or more sleep characteristics, how the exercise session should be customized. For example, if an REM threshold is met, then the difficulty may not be reduced, but if the REM threshold is not met, then the difficulty may be reduced by a static amount (e.g., by reducing the number of exercises or repetitions by ten percent) or a dynamic amount (e.g., by reducing the number of exercises or repetitions by a percentage that is based on the amount by which the REM threshold is missed). As another example, if multiple thresholds are missed—for example, an REM threshold and SWS threshold—then the difficulty may be reduced by an additive amount (e.g., ten percent for missing the REM threshold and another ten percent for missing the SWS threshold) or a combinative amount (e.g., ten percent for missing the REM threshold or SWS threshold, thirty percent for missing the REM threshold and SWS threshold). Said another way, the exercise session can be adjusted using a table that indicates how the exercises should be modified based on how the adjusted movement score—or the actual values for the sleep characteristics—compares to a threshold. This approach to quantifying the current physical state not only allows for appropriate customizations to be determined in a more computationally efficient manner, but also allows changes over time to be identified more easily. For example, the personalization module 322 may be able to observe upward or downward trends in physical state that might otherwise be undetectable.

In step 1210, the personalization module 322 can modify the exercise session based on the adjusted movement score computed in step 1208. For example, the personalization module 322 may exclude exercises with a difficulty level greater than the adjusted movement score and then indicate, in a data structure, that the user is “sleep deprived” if the good sleep condition has not been met. If the good sleep condition has been met, the personalization module 3222 may end any active customizations due to the user previously being marked as “sleep deprived.”

### Overview of Processing System

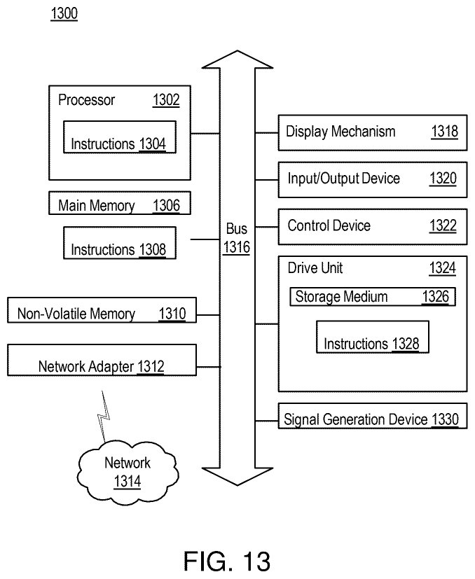

FIG. 13 includes a block diagram illustrating an example of a processing system 1300 in which at least some operations described herein can be implemented. For example, components of the processing system 1300 may be hosted on a computing device that includes a motion monitoring platform (e.g., motion monitoring platform 202 of FIG. 2 or motion monitoring platform 312 of FIG. 3).

The processing system 1300 can include a processor 1302, main memory 1306, non-volatile memory 1310, network adapter 1312, video display 1318, input/output devices 1320, control device 1322 (e.g., a keyboard or pointing device such as a computer mouse or trackpad), drive unit 1324 including a storage medium 1326, and signal generation device 1330 that are communicatively connected to a bus 1316. The bus 1316 is illustrated as an abstraction that represents one or more physical buses or point-to-point connections that are connected by appropriate bridges, adapters, or controllers. The bus 1316, therefore, can include a system bus, a Peripheral Component Interconnect (“PCI”) bus or PCI-Express bus, a HyperTransport (“HT”) bus, an Industry Standard Architecture (“ISA”) bus, a Small Computer System Interface (“SCSI”) bus, a Universal Serial Bus (“USB”) data interface, an Inter-Integrated Circuit (“I²C”) bus, or a high-performance serial bus developed in accordance with Institute of Electrical and Electronics Engineers (“IEEE”) 1394.

While the main memory 1306, non-volatile memory 1310, and storage medium 1326 are shown to be a single medium, the terms “machine-readable medium” and “storage medium” should be taken to include a single medium or multiple media (e.g., a centralized/distributed database and/or associated caches and servers) that store one or more sets of instructions 1328. The terms “machine-readable medium” and “storage medium” shall also be taken to include any medium that is capable of storing, encoding, or carrying a set of instructions for execution by the processing system 1300.

In general, the routines executed to implement the embodiments of the disclosure can be implemented as part of an operating system or a specific application, component, program, object, module, or sequence of instructions (collectively referred to as “computer programs”). The computer programs typically comprise one or more instructions (e.g., instructions 1304, 1308, 1328) set at various times in various memory and storage devices in a computing device. When read and executed by the processors 1302, the instruction(s) cause the processing system 1300 to perform operations to execute elements involving the various aspects of the present disclosure.

Further examples of machine- and computer-readable media include recordable-type media, such as volatile memory devices and non-volatile memory 1310, removable disks, hard disk drives, and optical disks (e.g., Compact Disk Read-Only Memory (“CD-ROMs”) and Digital Versatile Disks (“DVDs”)), and transmission-type media, such as digital and analog communication links.

The network adapter 1312 enables the processing system 1300 to mediate data in a network 1314 with an entity that is external to the processing system 1300 through any communication protocol supported by the processing system 1300 and the external entity. The network adapter 1312 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### REMARKS

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments can vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

1. A computing device comprising:
a communication module that is communicatively connectable to another computing device that is worn by a user;
a processor configured to:
retrieving sleep data from the other computing device in response to a determination that a temporal criterion is satisfied;
determining that the user does not satisfy a sleep condition based on an analysis of the sleep data; and
in response to said determining,
calculating a metric that is representative of quality of sleep, as determined based on the analysis of the sleep data; and
adjusting, based on the metric, a series of physical exercises to be performed by the user as part of an exercise session;
a display mechanism configured to present, to the user, a series of visualizations to guide performance of the adjusted series of physical exercises during the exercise session; and
an image sensor configured to generate digital images of the user as the adjusted series of physical exercises are performed.

2. The computing device of claim 1, wherein the processor is configured to determine that the temporal criterion is satisfied by comparing a clock signal to a predetermined time that is representative of the temporal criterion.

3. The computing device of claim 2, wherein the predetermined time is specified, by the user, through an interface presented by the display mechanism.

4. The computing device of claim 1, wherein the processor is configured to determine that the temporal criterion is satisfied by establishing, based on input received from a computer program executing on the computing device or an operating system of the computing device, that the user has awoken.

5. The computing device of claim 1, wherein said retrieving comprises forwarding, to the communication module, an instruction that is representative of a request for the sleep data for transmission to the other computing device.

6. The computing device of claim 1, wherein said adjusting comprises excluding at least one of the series of physical exercises that has a difficulty level greater than the metric.

7. The computing device of claim 1, wherein said adjusting comprises modifying at least one of the series of physical exercises the exercises by decreasing a repetition count or lessening a range of movement until a difficulty level is less than the metric.

8. A method performed by a computer program executing on a computing device, the method comprising:
retrieving sleep data that is associated with an individual who is to be guided, by the computer program, through a series of physical exercises as part of an exercise session;
determining whether the sleep data satisfies a predetermined criterion;
in response to a determination that the sleep data does satisfy the predetermined criterion,
causing a first series of visualizations to be presented, so as to guide performance of the series of physical exercises; and
in response to a determination that the sleep data does not satisfy the predetermined criterion,
adjusting, based on the sleep data, the series of physical exercises so as to decrease performance difficulty; and
causing a second series of visualizations to be presented, so as to guide performance of the adjusted series of physical exercises.

9. The method of claim 8, wherein the sleep data includes values for a plurality of sleep characteristics, and wherein said determining comprises establishing, for at least one of the plurality of sleep characteristics, whether a corresponding value satisfies a threshold.

10. The method of claim 8, wherein said determining comprises:
computing a value for a sleep characteristic based on an analysis of the sleep data, and
establishing whether the value satisfies a threshold.

11. The method of claim 10, wherein the sleep characteristic is rapid eye movement sleep, slow wave sleep, total sleep duration, eye movement, heart rate, respiratory rate, respiratory airflow, blood oxygen levels, body position, body movement, snoring presence, onset variance, or wake variance.

12. The method of claim 10, wherein said adjusting is accomplished using a table that indicates how to adjust the series of physical exercises based on how the value compares to the threshold.

13. The method of claim 8, further comprising:
in response to a determination that the sleep data does satisfy the predetermined criterion,
clearing any prior indication that the individual is sleep deprived from a data structure that is maintained for the individual.

14. The method of claim 8, further comprising:
in response to a determination that the sleep data does not satisfy the predetermined criterion,
indicating that the individual is sleep deprived in a data structure that is maintained for the individual.

15. The method of claim 8, wherein said determining comprises:
computing, based on an analysis of the sleep data, (i) a first value for a first sleep characteristic and (ii) a second value for a second sleep characteristic, and
establishing whether the first and second values satisfies respective thresholds.

16. The method of claim 15, wherein the first sleep characteristic is rapid eye movement sleep, and wherein the second sleep characteristic is slow wave sleep.
